(19) United States
(12) Patent Application Publication
(10) Pub. No.: US 2022/0117789 A1
(43) Pub. Date: Apr. 21, 2022

(54) LIGHT-PROOF EYE PROTECTION PASTE AND PREPARATION METHOD THEREOF

(71) Applicant: HANGZHOU JUJIU SCIENCE & BIOTECHNOLOGY CO.,, Hangzhou City (CN)

(72) Inventors: Chaofeng SHU, Hangzhou City (CN); Ruijun YU, Hangzhou City (CN); Fubo TIAN, Hangzhou City (CN)

(21) Appl. No.: 17/610,595

(22) PCT Filed: Nov. 20, 2020
(86) PCT No.: PCT/CN2020/130358

§ 371 Date: Nov. 11, 2021

(30) Foreign Application Priority Data
Dec. 4, 2019 (CN) 201911224828.4

(51) Int. Cl. A61F 9/04; A61N 5/067

(52) U.S. Cl. CPC A61F 9/04; A61N 5/067

(57) ABSTRACT

The disclosure provides a light-proof eye protection paste for preventing eyes from being damaged by light during phototherapy and a preparation method thereof, which solves problems of easy to fall off, skin burns, sticking off eyelashes, and provides safe and comfortable eye protection for phototherapy patients. The light-proof eye protection paste is composed of a decorative layer (1), a shading layer (2), a thermal insulating layer (3), a medical adhesive layer (4), an eyelash protective layer (5) and an isolation layer (6) from outside to inside, wherein the decorative layer (1) is located at the outermost layer for aesthetic decoration, the shading layer (2) is located inside the decorative layer (1) and used for blocking light and preventing light from penetrating to hurt the skin, the thermal insulating layer (3) is located inside the shading layer (2) and used for absorbing the strong light energy transmitted to the shading layer (2), the medical adhesive layer (4) is located inside the thermal insulating layer (3) and used for sticking and fixing on the skin around eyes, the eyelash protective layer (5) is located inside the medical adhesive layer (4) and used for protecting eyelashes from sticking off by the medical adhesive layer (4), and the isolation layer (6) is located inside the medical adhesive layer (4) and the eyelash protective layer (5), and is used for protecting the unused medical adhesive layer (4). The structure of the eye protection paste is compounded, cut and tailored by different methods to obtain the required products.

### TECHNICAL FIELD

The disclosure relates to a light-proof eye protection paste, in particular to a protective product capable of blocking the damage of light (laser/IPL/LED/blue light) to eyes during phototherapy.

### BACKGROUND ART

Various kinds of phototherapy (laser/IPL/LED/blue light) is widely used clinically, especially in the medical beauty. High-brightness light and high-energy light are prone to hurt eyes, which may lead to photophobia, tears, hypopsia or blindness. According to data, it is showed that an irradiation dose causing fundus macular lesion is 10 mw/cm², and an irradiation time is only picoseconds, and the energy density of intense pulsed light/laser is generally between 100 mw and 200 mw/cm². Therefore, during various kinds of phototherapy, doctors and patients need to be equipped with professional protective facilities to avoid medical accidents.

At present, the main protective products for patients with various kinds of phototherapy are laser protective eye patch and laser protective eye paste. Laser eye patch is generally made of flexible opaque plastic and fixed on the back of the brain with fixed rope. The laser protective eye paste is fixed with adhesive. However, the laser eye patch has the following defects: 1. after the laser eye patch is worn on the eyes, there may be a gap between the edge of the patch and the skin, and the light may hurt the eyes through the gap; 2. when the patient is in a supine position during an operation, the fixing rope of the laser eye patch are contacted with the pillow or the operating table (such as the laser protective eye patch mentioned in Patent CN 201821262093.5), and a slight movement of the head may cause the eye patch to move or fall off, which may cause the eyes to be exposed to the light and be damaged by strong light. A laser protective eye paste, as mentioned in JP2007160023A, is a product consisting of a substrate, a first adhesive layer, a non-woven fabric, a second adhesive layer, and an isolation film. There is a defect in the design of the product, when the laser irradiates on the eye protection paste, although the eye protection paste can prevent the direct damage of light to the eyes, the heat generated by its high energy will easily cause eye skin burn (New regulations have been issued in Europe that laser protection products must have the function of preventing skin burns). Chinese patent 201310073280.4 provides a hydrogel laser eye protective paste, which is composed of three layers: a support layer, a hydrogel layer and an isolation layer. The hydrogel layer is in contact with the skin, and the hydrogel has soft viscosity and could protect patients' eyelashes or eyebrow after removal, but its adhesion with the skin is too weak to be firmly fixed on eyelid skin during clinical use, so it is easy to fall off, resulting in increased risk of eye injury during photon/laser surgery. Chinese patent 206026537 provides a laser eye paste composed of a shading part (including a supporting layer, a heat absorbing layer and a reflecting layer), an adhesive part and a release paper. The shading part is designed with a convex structure to improve laser reflectivity and avoid discomfort caused by compression, and a vibration structure is arranged in the eye paste to massage eye muscles and relieve psychological pressure. However, the design has the following defects: 1. the laser can directly interact with the heat absorbing layer, causing burning effects and the like, and the generated heat will be directly transmitted to the skin through the reflecting layer, causing thermal damage to the skin; 2. the reflecting layer is arranged behind the heat absorbing layer, which can prevent the laser from directly damaging the eyes, but after the laser is reflected, it reacts with the heat absorbing layer again, and the laser energy converts into heat energy of the layer again, which will increase the risk of thermal damage to the skin of the eyes; 3. the laser cauterizes the supporting layer and the heat absorbing layer, which may produce smoke to affect the operator's vision and pollute the environment, thereby harming the health of the operators and the patients.

U.S. Pat. No. 7,584,754B1 provides a disposable eye patch composed of a first layer and a second layer, which are made of biocompatible foamed plastic material, and an isolation layer, wherein the first layer and the second layer are bonded together by an adhesive layer made of pressure sensitive adhesive, and the second layer is smaller than the first layer, and the product is adhered to the skin around the patient's eyes by an adhesive layer exposed outside the second layer. This patent product is suitable for eye protection under non strong light/laser conditions. High-energy light such as laser can easily penetrate the product and hurt the eyes, thus failing to protect the eyes.

Therefore, the purpose of the present disclosure is to develop a shading product with the advantages of simple structure, simple production process, effective prevention of laser/strong light injury to patients' eyes, convenient to use, good compatibility, firm and safe fixation and more comfortable experience for patients.

### SUMMARY

The objective of the disclosure is to overcome the deficiencies of the prior art and the problems in the clinical application process: 1. the product is not firmly fixed on the eyelid skin and is easy to fall off; 2. when high-energy light such as laser irradiates the surface of the product, the temperature of the product rises, resulting in the eyelid skin burns; 3. during the use of the product, the product may hurt the skin and its affiliated organs (eyelashes); 4. when being irradiated with strong light, the product produces toxic smoke, which affects vision and health. Therefore, in order to provide a safe, effective and comfortable eye protection paste for clinic, the present disclosure provides the following technical scheme:

The light-proof eye protection paste consists of a decorative layer, a shading layer, a thermal insulating layer, a medical adhesive layer, an eyelash protective layer and an isolation layer from outside to inside, wherein the decorative layer is located at the outermost layer for aesthetic decoration, the shading layer is located inside the decorative layer and used for blocking light and preventing light from penetrating to hurt the skin, the medical adhesive layer is located inside the thermal insulating layer and used for sticking and fixing on the skin around eyes, the eyelash protective layer is located inside the medical adhesive layer and used for protecting eyelashes from sticking off by the medical adhesive layer, and the isolation layer is located inside the medical adhesive layer and the eyelash protective layer, and is used for protecting the unused medical adhesive layer.

In some embodiments, the decorative layer is a film with a thickness in a range of 0.01-0.5 mm, preferably in a range of 0.02-0.05 mm, and a color of the film can be any color, preferably transparent or translucent, but not limited to the above colors. The film can be fixed on the outer surface of the shading layer by general means (laminating or glue bonding process). The film can be made of PU, PE, PP and the like, but is not limited to the above materials.

In some embodiments, the shading layer is a metal foil, and the metal foil can include any metal capable of forming a foil, preferably aluminum foil or copper foil.

In some embodiments, the shading layer is a metallized film, preferably an aluminized film.

In some embodiments, the shading layer has a thickness in a range of 0.01-0.2 mm, preferably in a range of 0.05-0.15 mm.

In some embodiments, the thermal insulating layer is a thermal poor conductor, with porous structure and soft texture, preferably foam or sponge structure.

In some embodiments, the thermal insulating layer is a thermal poor conductor, with film-like structure and soft texture.

In some embodiments, a material of the thermal insulating layer may be selected from one of polyurethane (PU), ethylene-propylene-diene monomer (EPDM), expandedpolypropylene (EPP), ethylene-vinyl acetate copolymer (EVA), chemical crosslinked polyethylene (XPE)/electron crosslinked polyethylene (IXPE) foam, with a thickness of 0.1-1.5 mm, preferably 0.5-0.8 mm. In the foam structure, the foam is preferably a compact foam with two smooth sides. The thermal insulating layer can be made of foam, but not limited to foam. Any material with characteristics of soft, safe and poor thermal conductivity, but not limited to, will do, such as sponge.

In some embodiments, the thermal insulating layer is made of a non-transparent material, which plays a certain role in blocking light at the same time of heat insulation, during IPL/LED skin treatment/blue light treatments of neonatal jaundice with low/no heat, the shading layer can be removed and the thermal insulating layer is used directly as the shading layer to block light. Due to the skin thermal damage caused by the blue light (a wavelength of 400-550 nm) used in IPL/LED or neonatal jaundice treatment is lower than that of laser, if the material and structure used in the thermal insulating layer can completely block the penetration of IPL/LED/blue light, the shading layer can be omitted, which can save the production cost and simplify the production process, and is beneficial to the industrialization of products.

In some embodiments, the thermal insulating layer has a dual structure composed of a smooth foam layer and a hydrogel layer, the smooth foam layer is in contact with the shading layer, and the hydrogel layer replaces the medical adhesive layer to adhere to the skin. In one embodiment, the thermal insulating layer is composed of a foam or film layer with poor thermal conductivity and a hydrogel layer with large specific heat capacity, which has stronger thermal insulation effect. Since the adhesive force of the hydrogel layer is limited, in another embodiment, a composite film composed of a non-woven fabric and an organic film is added inside the hydrogel layer, and the medical adhesive is coated on the other side of the organic film of the composite film to adhere to the skin and increase its stability.

In some embodiments, the decorative layer film and the thermal insulating layer are fixed on both sides of the shading layer by laminating process.

In some embodiments, the decorative layer and the thermal insulating layer are compounded on both sides of the shading layer by the following steps: 1) keeping the film of the decorative layer and materials of the shading layer even and non-folding under proper tension conditions; 2) coating a polyurethane adhesive on the surface of the decorative layer, after natural drying for 1-3 minutes, and compounding the decorative layer with the shading layer under the extrusion of a pressure roller; 3) repeating the above steps 1) and 2), and compounding the thermal insulating layer on the other side of the shading layer; 4) curing the polyurethane adhesive between the decorative layer, the shading layer and the thermal insulating layer for 5-36 hours under conditions of temperature 35-60° C. and humidity 35-60% to complete the compounding process.

In some embodiments, the medical adhesive layer comprises a safe, hypoallergenic and biocompatible medical adhesive, which can be one of medical hot-melt pressure-sensitive adhesive, acrylic pressure-sensitive adhesive and silicone gel, preferably acrylic pressure-sensitive adhesive.

In some embodiments, the eyelash protective layer is a soft, non-sticky, oval or round, biocompatible material with a thickness of 0.01-0.2 mm, such as polyethylene (PE), preferably a film.

In some embodiments, the eyelash protective layer is located at the center of the medical adhesive layer, and the edge area of the medical adhesive layer not covered by the eyelash protective layer has an enough width to fix the paste on the skin, the width is usually in a range of 3-15 mm, preferably 5 mm. The structure of this part is mainly used to protect eyelashes and prevent eyelashes from being removed by medical adhesive. The part of the medical adhesive layer that is not covered by the eyelash protective layer is in contact with the skin to fix the product on the skin.

In some embodiments, the isolation layer is selected from one of silicone oil release paper, non-silicon release paper, fluorinated PET film, silicone oil PET film and PET film. If the medical adhesive layer material is silicone gel, the isolation layer can be selected from one of non-silicon release paper, fluorinated PET film and PET film, and the silicone oil release paper and silicone oil PET film can not be used.

In some embodiments, the shape and shading layer of the paste is conforming to the structure and physiological characteristics of the eyes, and can be selected from one of symmetrical rain drop shape, bean sprout shape, oval shape, dolphin shape or conjoined glasses frame shape, but not limited to the above shapes, preferably bean sprout shape.

In some embodiments, the light-proof eye protection paste is symmetrically arranged and adhered on the rectangular isolation layer, and is separately adhered on the rectangular isolation layer when it is in the shape of a conjoined glasses frame.

The method for preparing the light-proof eye protection paste is as follows:

1) preparing a composite material by using the decorative layer, shading layer, thermal insulating layer, medical adhesive layer and eyelash protective layer according to the above method, and rolling the material into a coil, the shading layer is close to the heading when being rolled;

2) cutting off the eyelash protective layer of the coil with an oval or round semi-cutter (which can exactly cut off the eyelash protective layer without cutting the medical adhesive layer), peeling off the extra eyelash protective layer outside the central oval or round from the medical adhesive layer through a material receiving system, and compounding the exposed medical adhesive layer and the isolation layer together;

3) for the compounded material obtained in step 2), using the semi-cutter with the shape of claim 18, adjusting the position (ensuring that cutting off the central position of the profile in step 3) after cutting off the eyelash protective layer adhered to the medical adhesive layer in step 2)), cutting off the the decorative layer, shading layer, thermal insulating layer and medical adhesive layer without cutting the isolation layer, leaving the composite material with specific shape on the surface of the isolation layer, and peeling off other materials from the isolation layer through the material receiving system, and then cutting the isolation layer by the semi-cutter to obtain the product.

By adopting the above scheme, the light-proof eye protection paste of the disclosure has the advantages of simple structure, convenient production, good fixation performance and biocompatibility, and is able to avoid skin burns, eyelash damages, thus providing a safe, effective and comfortable light-proof eye protection paste for clinic, and the product can be stored for a long time after being sterilized.

The disclosure has the following beneficial effects:

1. The technical scheme of the present disclosure has the advantages of proper adhesion, firm fixation, no shedding, and avoiding tearing of eyelashes of patients.

2. The technical scheme of the disclosure is able to protect the eyes of patients from being hurt by the light and heat during treatment.

3. There is little or no smoke produced when being irradiated by strong light, which has little influence on the environment and visual field.

4. The shape of the paste in the disclosure is designed according to the principle of ergonomics, which accords with various physiological bends of local eyes, is convenient to use and has good adhesion effect.

5. The materials adopted in the disclosure have high safety and good biocompatibility.

6. The product has the characteristics of simple structure, convenient production and low cost.

### BRIEF DESCRIPTION OF THE DRAWINGS

FIG. 1 is a sectional view of the light-proof eye protection paste of the present disclosure;

where, 1 represents the decorative layer, 2 represents the shading layer, 3 represents the thermal insulating layer, 4 represents the medical adhesive layer, 5 represents the eyelash protective layer, 6 represents the isolation layer, 7 represents the hydrogel layer, 8 represents the non-woven fabric layer and 9 represents the organic film.

FIG. 2 is a vertical view of the first and sixth example of the present disclosure.

FIG. 3 is a vertical view of the second example of the present disclosure.

FIG. 4 is a vertical view of the third example of the present disclosure.

FIG. 5 is a vertical view of the fourth and fifth example of the present disclosure.

FIG. 6 is a vertical view of the seventh example of the present disclosure.

FIG. 7 is a sectional view of the fifth example of the present disclosure.

FIG. 8 is a sectional view of the sixth example of the present disclosure.

FIG. 9 is a sectional view of the seventh example of the present disclosure.

### DETAILED DESCRIPTION OF THE EMBODIMENTS

The disclosure will be further explained with examples below:

### Example 1

As shown in FIG. 1, a light-proof eye protection paste was composed of a decorative layer, a shading layer, a thermal insulating layer, a medical adhesive layer, an eyelash protective layer and an isolation layer, wherein the decorative layer was located at the outermost layer for aesthetic decoration, which was a translucent milky white film on the surface of the shading layer with a thickness of 0.02 mm; the shading layer was located inside the decorative layer and used for blocking light and preventing light from penetrating to hurt the skin, and was made of metal aluminum foil with a thickness of 0.07 mm. The thermal insulating layer was located inside the shading layer and used for absorbing the strong light energy transmitted to the shading layer, which was a non-transparent XPE foam with a thickness of 0.5 mm.

The decorative layer, the shading layer and the thermal insulating layer were compounded together by the following steps: 1) the decorative layer and the shading layer were kept even and non-folding under proper tension conditions; 2) a polyurethane adhesive was coated on the surface of the shading layer, after natural drying for 2 minutes, the shading layer was compounded with the thermal insulating layer under the extrusion of a pressure roller; 3) for the compounded materials, the polyurethane adhesive between the shading layer and the thermal insulating layer was cured for 12 hours under conditions of temperature 55° C. and humidity 45% to complete the compounding process. The shading layer of the compound of the decorative layer and the shading layer and the thermal insulating layer were compounded through the same process described above. The compound process of the decorative layer, the shading layer and the thermal insulating layer was completed by performing the above process twice.

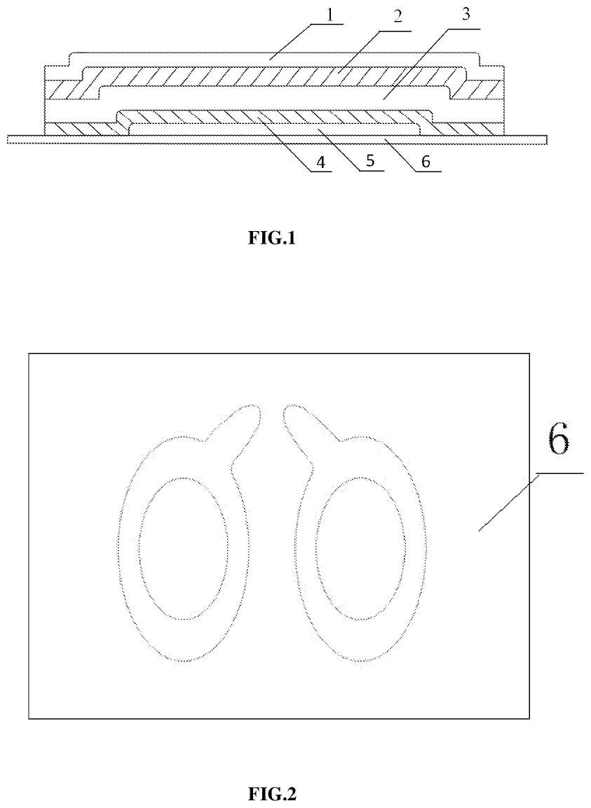

The medical adhesive layer, which was acrylic pressure-sensitive adhesive, was located inside the thermal insulating layer and used for sticking and fixing on the skin around eyes. The eyelash protective layer was located inside the medical adhesive layer and used for protecting eyelashes from removing by the medical adhesive layer, which was a non-sticky PE film with a thickness of 0.04 mm and had an area smaller than that of the medical adhesive layer. The distance between the outer edge of the medical adhesive layer and the outer edge of the eyelash protective layer was 5 mm. The isolation layer was located inside the medical adhesive layer and the eyelash protective layer, which was used to protect the unused medical adhesive layer and was silicone oil release paper. The shape of the light-proof eye protection pastes was bean sprout, and the light-proof eye protection pastes were symmetrically arranged and adhered on rectangular isolation paper, as shown in FIG. 2.

The product was completed by the following steps: 1) a composite material was prepared by using the decorative layer, shading layer, thermal insulating layer, medical adhesive layer and eyelash protective layer according to the above method, and the material was rolled into a coil, when being rolled, the shading layer was close to the heading; 2) the eyelash protective layer of the coil was cut off with an oval semi-cutter (which can exactly cut off the eyelash protective layer without cutting the medical adhesive layer), the extra eyelash protective layer outside the central oval was peeled off from the medical adhesive layer through a material receiving system, and the exposed medical adhesive layer and the isolation layer were compounded together; 3) for the compounded material obtained in step 2), a semi-cutter with the shape of bean sprout was used, the position (ensuring that cutting off the central position of the profile in step 3) after cutting off the eyelash protective layer adhered to the medical adhesive layer in step 2)) was adjusted to cut off the decorative layer, shading layer, thermal insulating layer and medical adhesive layer without cutting the isolation layer, the composite material with bean sprout shape was left on the surface of the isolation layer, and other materials were peeled off from the isolation layer through the material receiving system, and then the isolation layer was cut by the semi-cutter to obtain the product.

### Example 2

As shown in FIG. 1, a light-proof eye protection paste was composed of a decorative layer, a shading layer, a thermal insulating layer, a medical adhesive layer, an eyelash protective layer and an isolation layer, wherein the decorative layer was located at the outermost layer for aesthetic decoration, which was a transparent blue film with a thickness of 0.01 mm; the shading layer was located inside the decorative layer and used for blocking light and preventing light from penetrating to hurt the skin, and was made of aluminized film with a thickness of 0.2 mm. The thermal insulating layer was located inside the shading layer and used for absorbing the strong light energy transmitted to the shading layer, which was a non-transparent IXPE foam with a thickness of 0.7 mm. The decorative layer and the shading layer were compounded by a conventional laminating process.

The decorative layer, the shading layer and the thermal insulating layer were compounded together by the following steps: 1) the film of the decorative layer/the compound of the shading layer and the materials of the thermal insulating layer were kept even and non-folding under proper tension conditions; 2) a polyurethane adhesive was coated on the surface of the decorative layer, after natural drying for 1 minute, the decorative layer was compounded with the shading layer under the extrusion of a pressure roller; 3) the compounded materials was cured for 36 hours under conditions of temperature 35° C. and humidity 45% to complete the compounding process. The shading layer of the compound of the decorative layer and the shading layer and the thermal insulating layer were compounded through the same process described above. The compound process of the decorative layer, the shading layer and the thermal insulating layer was completed by performing the above process twice.

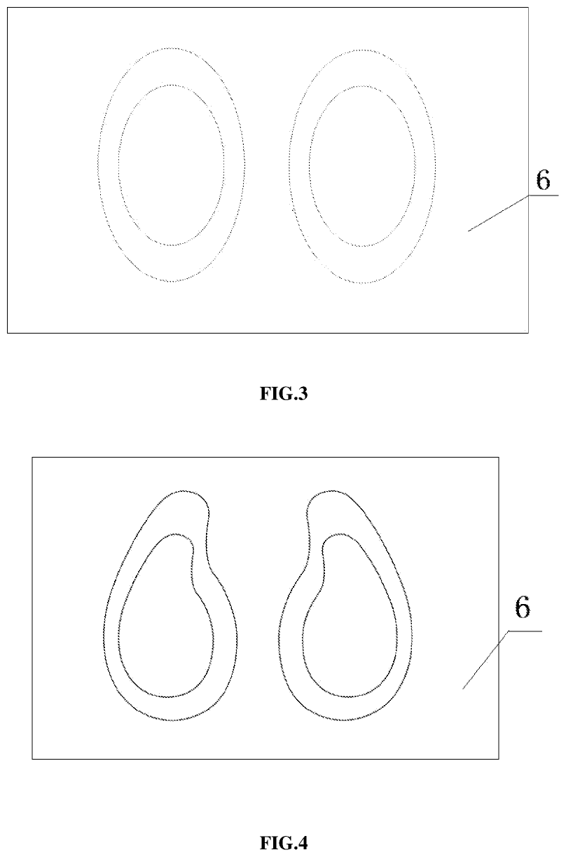

The medical adhesive layer, which was silicon gel, was located inside the thermal insulating layer and used for sticking and fixing on the skin around eyes. The eyelash protective layer was located inside the medical adhesive layer and used for protecting eyelashes from sticking off by the medical adhesive layer, which was a non-sticky PP (polypropylene) film with a thickness of 0.01 mm and had an area smaller than that of the medical adhesive layer. The distance between the outer edge of the medical adhesive layer and the outer edge of the eyelash protective layer was 3 mm. The isolation layer, which was a fluorinated PET film, was located inside the medical adhesive layer and the eyelash protective layer, and was used to protect the unused medical adhesive layer. The shape of the light-proof eye protection paste was oval, and the light-proof eye protection paste was symmetrically arranged and adhered to the rectangular fluorinated PET film, as shown in FIG. 3.

The product was completed by the following steps: 1) a composite material was prepared by using the decorative layer, shading layer, thermal insulating layer, medical adhesive layer and eyelash protective layer according to the above method, and the material was rolled into a coil, when being rolled, the shading layer was close to the heading; 2) the eyelash protective layer of the coil was cut off with an oval semi-cutter (which can exactly cut off the eyelash protective layer without cutting the medical adhesive layer), the extra eyelash protective layer outside the central oval was peeled off from the medical adhesive layer through a material receiving system, and the exposed medical adhesive layer and the isolation layer were compounded together; 3) for the compounded material obtained in step 2), a semi-cutter with the oval shape was used, the position (ensuring that cutting off the central position of the profile in step 3) after cutting off the eyelash protective layer adhered to the medical adhesive layer in step 2)) was adjusted to cut off the decorative layer, shading layer, thermal insulating layer and medical adhesive layer without cutting the isolation layer, the composite material with oval shape was left on the surface of the isolation layer, and other materials were peeled off from the isolation layer through the material receiving system, and then the isolation layer was cut by the semi-cutter to obtain the product.

### Example 3

As shown in FIG. 1, a light-proof eye protection paste was composed of a decorative layer, a shading layer, a thermal insulating layer, a medical adhesive layer, an eyelash protective layer and an isolation layer, wherein the decorative layer was located at the outermost layer for aesthetic decoration, which was a translucent blue film on the surface of the shading layer; the shading layer was located inside the decorative layer and used for blocking light and preventing light from penetrating to hurt the skin, and was made of metal aluminum foil with a thickness of 0.05 mm. The thermal insulating layer was located inside the shading layer and used for absorbing the strong light energy transmitted to the shading layer, which was a non-transparent PE film with a thickness of 0.1 mm.

The decorative layer, the shading layer and the thermal insulating layer were compounded together by the following steps: 1) the materials of the decorative layer and the shading layer were kept even and non-folding under proper tension conditions; 2) a polyurethane adhesive was coated on the surface of the decorative layer, after natural drying for 2 minutes, and the shading layer was compounded with the thermal insulating layer under the extrusion of a pressure roller; 3) for the compounded materials, the polyurethane adhesive between the decorative layer and the shading layer was cured for 5 hours under conditions of temperature 60° C. and humidity 60% to complete the compounding process. As for the compound of the decorative layer and the shading layer, the other side of the shading layer was compounded with the thermal insulating layer through the above same process. The compound process of the decorative layer, the shading layer and the thermal insulating layer was completed by performing the above process twice.

The medical adhesive layer, which was a medical hot-melt pressure-sensitive adhesive, was located inside the thermal insulating layer and used for sticking and fixing on the skin around eyes. The eyelash protective layer was located inside the medical adhesive layer and used for protecting eyelashes removal by the medical adhesive layer, which was a non-sticky PU film with a thickness of 0.2 mm and had an area smaller than that of the medical adhesive layer. The distance between the outer edge of the medical adhesive layer and the outer edge of the eyelash protective layer was 15 mm. The isolation layer was located inside the medical adhesive layer and the eyelash protective layer to protect the unused medical adhesive layer and was silicone oil release paper. The shape of the light-proof eye protection pastes was drop-shaped, and the light-proof eye protection pastes were symmetrically arranged and adhered on rectangular isolation paper, as shown in FIG. 4.

The product was completed by the following steps: 1) a composite material was prepared by using the decorative layer, shading layer, thermal insulating layer, medical adhesive layer and eyelash protective layer according to the above method, and the material was rolled into a coil, when being rolled, the shading layer was close to the heading; 2) the eyelash protective layer of the coil was cut off with a drop-shaped semi-cutter (which can exactly cut off the eyelash protective layer without cutting the medical adhesive layer), the extra eyelash protective layer outside the drop-shaped area was peeled off from the medical adhesive layer through a material receiving system, and the exposed medical adhesive layer and the isolation layer were compounded together; 3) for the compounded material obtained in step 2), a semi-cutter with the drop shape was used, the position (ensuring that cutting off the central position of the profile in step 3) after cutting off the eyelash protective layer adhered to the medical adhesive layer in step 2)) was adjusted to cut off the decorative layer, shading layer, thermal insulating layer and medical adhesive layer without cutting the isolation layer, the composite material with drop shape was left on the surface of the isolation layer, and other materials were peeled off from the isolation layer through the material receiving system, and then the isolation layer was cut by the semi-cutter to obtain the product.

### Example 4

As shown in FIG. 1, a light-proof eye protection paste was composed of a decorative layer, a shading layer, a thermal insulating layer, a medical adhesive layer, an eyelash protective layer and an isolation layer, wherein the decorative layer was located at the outermost layer for aesthetic decoration, which was a black film on the surface of the shading layer with a thickness of 0.03 mm; the shading layer was located inside the decorative layer and used for blocking light and preventing light from penetrating to hurt the skin, and was made of metal aluminum foil with a thickness of 0.09 mm. The thermal insulating layer was located inside the shading layer and used for absorbing the strong light energy transmitted to the shading layer, which was a non-transparent EVA foam with a thickness of 1.5 mm.

The decorative layer, the shading layer and the thermal insulating layer were compounded together by the following steps: 1) the decorative layer and the shading layer were kept even and non-folding under proper tension conditions; 2) a polyurethane adhesive was coated on the surface of the decorative layer, after natural drying for 2 minutes, and the shading layer was compounded with the thermal insulating layer under the extrusion of a pressure roller; 3) for the compounded materials, the polyurethane adhesive between the decorative layer and the shading layer was cured for 18 hours under conditions of temperature 50° C. and humidity 40% to complete the compounding process. As for the compound of the decorative layer and the shading layer, the other side of the shading layer was compounded with the thermal insulating layer through the above same process. The compound process of the decorative layer, the shading layer and the thermal insulating layer was completed by performing the above process twice.

The medical adhesive layer, which was acrylic pressure-sensitive adhesive, was located inside the thermal insulating layer and used for sticking and fixing on the skin around eyes. The eyelash protective layer was located inside the medical adhesive layer and used for protecting eyelashes from sticking off by the medical adhesive layer, which was a non-sticky PE (polyethylene) film with a thickness of 0.05 mm and had an area smaller than that of the medical adhesive layer. The distance between the outer edge of the medical adhesive layer and the outer edge of the eyelash protective layer was 8.5 mm. The isolation layer was located inside the medical adhesive layer and the eyelash protective layer, which was used to protect the unused medical adhesive layer and was non-silicon release paper. The shape of the light-proof eye protection paste was glasses shape, and the light-proof eye protection paste was adhered to rectangular isolation paper, as shown in FIG. 5.

The product was completed by the following steps: 1) a composite material was prepared by using the decorative layer, shading layer, thermal insulating layer, medical adhesive layer and eyelash protective layer according to the above method, and the material was rolled into a coil, when being rolled, the shading layer was close to the heading; 2) the eyelash protective layer of the coil was cut off with a glasses shape semi-cutter (which can exactly cut off the eyelash protective layer without cutting the medical adhesive layer), the extra eyelash protective layer outside the glasses shape area was peeled off from the medical adhesive layer through a material receiving system, and the exposed medical adhesive layer and the isolation layer were compounded together; 3) for the compounded material obtained in step 2), a semi-cutter with the glasses shape was used, the position (ensuring that cutting off the central position of the profile in step 3) after cutting off the eyelash protective layer adhered to the medical adhesive layer in step 2)) was adjusted to cut off the decorative layer, shading layer, thermal insulating layer and medical adhesive layer without cutting the isolation layer, the composite material with glasses shape was left on the surface of the isolation layer, and other materials were peeled off from the isolation layer through the material receiving system, and then the isolation layer was cut by the semi-cutter to obtain the product.

### Example 5

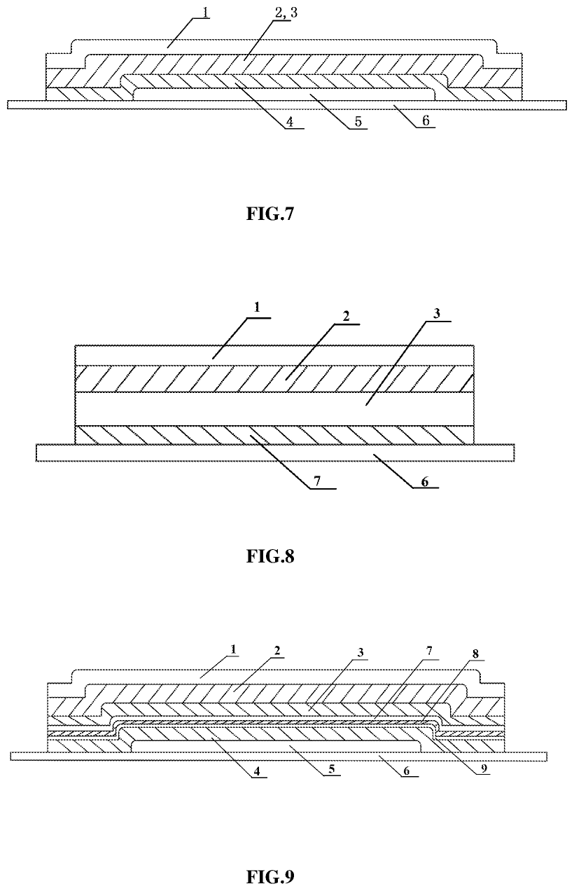

As shown in FIG. 7, a light-proof eye protection paste was composed of a decorative layer, a shading/thermal insulating layer, a medical adhesive layer, an eyelash protective layer and an isolation layer, wherein the decorative layer was located at the outermost layer for aesthetic decoration, which was a translucent milky white film on the surface of the shading layer with a thickness of 0.01 mm; the shading layer and the thermal insulating layer were combined into one layer and located inside the decorative layer, using for blocking light and preventing light from penetrating to hurt the skin, which was a non-transparent EPDM foam with a thickness of 0.6 mm.

The decorative layer, the EPDM foam were compounded together by the following steps: 1) the decorative layer and the EPDM foam were kept even and non-folding under proper tension conditions; 2) a polyurethane adhesive was coated on the surface of the EPDM foam, after natural drying for 2 minutes, and the EPDM foam was compounded with the thermal insulating layer under the extrusion of a pressure roller; 3) for the compounded materials, the polyurethane adhesive between the shading layer and the thermal insulating layer was cured for 14 hours under conditions of temperature 55° C. and humidity 45% to complete the compounding process.

The medical adhesive layer, which was acrylic pressure-sensitive adhesive, was located inside the thermal insulating layer and used for sticking and fixing on the skin around eyes. The eyelash protective layer was located inside the medical adhesive layer and used for protecting eyelashes from sticking off by the medical adhesive layer, which was a non-sticky PE (polyethylene) film with a thickness of 0.04 mm and had an area smaller than that of the medical adhesive layer. The distance between the outer edge of the medical adhesive layer and the outer edge of the eyelash protective layer was 15 mm. The isolation layer was located inside the medical adhesive layer and the eyelash protective layer, which was used to protect the unused medical adhesive layer and was a PET film. The shape of the light-proof eye protection paste was glasses shape, and the light-proof eye protection paste was adhered to rectangular isolation paper, as shown in FIG. 5.

The product was completed by the following steps: 1) a composite material was prepared by using the decorative layer, shading layer, thermal insulating layer, medical adhesive layer and eyelash protective layer according to the above method, and the material was rolled into a coil, when being rolled, the shading layer was close to the heading; 2) the eyelash protective layer of the coil was cut off with a glasses shape semi-cutter (which can exactly cut off the eyelash protective layer without cutting the medical adhesive layer), the extra eyelash protective layer outside the glasses shape area was peeled off from the medical adhesive layer through a material receiving system, and the exposed medical adhesive layer and the isolation layer were compounded together; 3) for the compounded material obtained in step 2), a semi-cutter with the glasses shape was used, the position (ensuring that cutting off the central position of the profile in step 3) after cutting off the eyelash protective layer adhered to the medical adhesive layer in step 2)) was adjusted to cut off the decorative layer, shading layer, thermal insulating layer and medical adhesive layer without cutting the isolation layer, the composite material with glasses shape was left on the surface of the isolation layer, and other materials were peeled off from the isolation layer through the material receiving system, and then the isolation layer was cut by the semi-cutter to obtain the product.

The light-proof eye protection paste of this example was mainly used for eye protection during low-energy light treatment except laser, such as IPL and LED medical cosmetology, and can also be used for eye protection of children during blue light treatment of neonatal jaundice.

### Example 6

As shown in FIG. 8, a light-proof eye protection paste was composed of a decorative layer, a shading layer, a thermal insulating layer, a hydrogel layer and an isolation layer, wherein the decorative layer was located at the outermost layer for aesthetic decoration, which was a translucent frosted film on the surface of the shading layer with a thickness of 0.02 mm; the shading layer was located inside the decorative layer and used for blocking light and preventing light from penetrating to hurt the skin, and was made of metal aluminum foil with a thickness of 0.09 mm. The thermal insulating layer was located inside the shading layer and used for absorbing the strong light energy transmitted to the shading layer, which was a dual structure composted of non-transparent EPP foam and hydrogel, the non-transparent EPP foam had a thickness of 0.6 mm.

The decorative layer, the shading layer and the EPP foam were compounded together by the following steps: 1) the decorative layer and the shading layer were kept even and non-folding under proper tension conditions; 2) a polyurethane adhesive was coated on the surface of the decorative layer, after natural drying for 2 minutes, and the shading layer was compounded with the decorative layer under the extrusion of a pressure roller; 3) for the compounded materials, the polyurethane adhesive between the decorative layer and the shading layer was cured for 12 hours under conditions of temperature 55° C. and humidity 45% to complete the compounding process. As for the compound of the decorative layer and the shading layer, the other side of the shading layer was compounded with the EPP foam through the above same process. The compound process of the decorative layer, the shading layer and the EPP foam was completed by performing the above process twice.

After compounding, the rough surface of EPP foam was contacted with the hydrogel, namely, the hydrogel was directly coated on EPP foam. The other side of hydrogel was an isolation layer, and the isolation layer was silicone oil PET film. The shape of the light-proof eye protection paste was bean sprout, and the light-proof eye protection paste was adhered to rectangular isolation paper, as shown in FIG. 2.

The product was completed by the following steps: 1) a composite material was prepared by using the decorative layer, shading layer, hydrogel layer, and isolation layer according to the above method, and the material was rolled into a coil, when being rolled, the shading layer was close to the heading; 2) the the decorative layer, shading layer, hydrogel layer of the composite material was cut off with a bean sprout shape semi-cutter without cutting the isolation layer, the composite material with bean sprout shape was left on the surface of the isolation layer, and other materials were peeled off from the isolation layer through the material receiving system, and then the isolation layer was cut through the semi-cutter to obtain the product.

The thermal insulating layer of the light-proof eye protection paste in this example had a dual structure of EPP foam and hydrogel, which has a better thermal insulation effect than that of a single-layer foam thermal insulating layer. For the strong light (such as laser) that can produce high heat effect, it has better protection and avoids skin scald.

### Example 7

As shown in FIG. 9, a light-proof eye protection paste was composed of a decorative layer, a shading layer, a thermal insulating layer, a hydrogel layer, a non-woven fabric, an organic film layer, a medical adhesive layer, an eyelash protective layer and an isolation layer, wherein the decorative layer was located at the outermost layer for aesthetic decoration, which was a translucent milky white film on the surface of the shading layer with a thickness of 0.02 mm; the shading layer was located inside the decorative layer and used for blocking light and preventing light from penetrating to hurt the skin, and was made of metal aluminum foil with a thickness of 0.07 mm. The thermal insulating layer was located inside the shading layer and used for absorbing the strong light energy transmitted to the shading layer, which was a dual structure composted of non-transparent IXPE foam and hydrogel, the IXPE foam had a thickness of 0.4 mm.

The decorative layer, the shading layer and the IXPE foam were compounded together by the following steps: 1) the decorative layer and the shading layer were kept even and non-folding under proper tension conditions; 2) a polyurethane adhesive was coated on the surface of the decorative layer, after natural drying for 2 minutes, and the shading layer was compounded with the decorative layer under the extrusion of a pressure roller; 3) for the compounded materials, the polyurethane adhesive between the decorative layer and the shading layer was cured for 12 hours under conditions of temperature 55° C. and humidity 45% to complete the compounding process. As for the compound of the decorative layer and the shading layer, the other side of the shading layer was compounded with the IXPE foam through the above same process. The compound process of the decorative layer, the shading layer and the IXPE foam was completed by performing the above process twice.

After compounding, the IXPE foam surface of the material was contacted with the hydrogel, namely, the hydrogel was directly coated on IXPE foam. The other side of the hydrogel was a composite material of non-woven fabric and organic film (PE film). The non-woven fabric surface of the non-woven fabric/PE film composite was contacted with the hydrogel, and the other side of PE film was coated with medical adhesive.

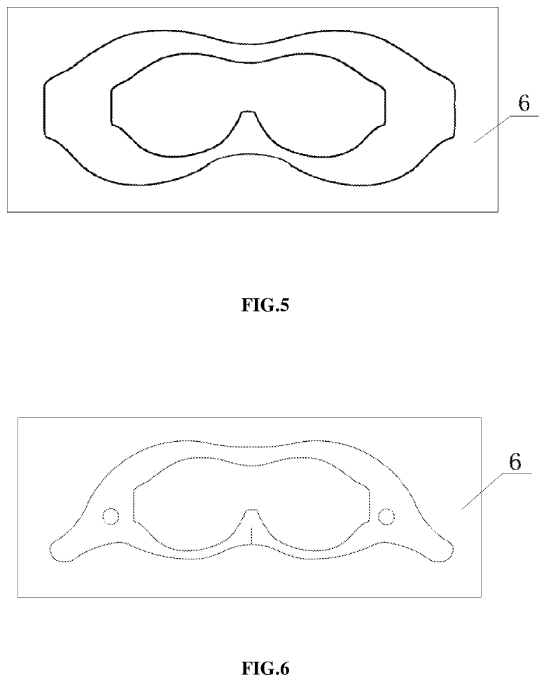

The medical adhesive layer, which was acrylic pressure sensitive adhesive, was located inside the PE film and used for sticking and fixing on the skin around eyes. The eyelash protective layer was located inside the medical adhesive layer and used for protecting eyelashes from sticking off by the medical adhesive layer, which was a non-sticky PE (polyethylene) frosted film with a thickness of 0.08 mm and had an area smaller than that of the medical adhesive layer. The distance between the outer edge of the medical adhesive layer and the outer edge of the eyelash protective layer was 7 mm. The isolation layer was located inside the medical adhesive layer and the eyelash protective layer to protect the unused medical adhesive layer and was silicone oil release paper. The shape of the light-proof eye protection paste was dolphin-shaped, and the light-proof eye protection paste was adhered to rectangular isolation paper, as shown in FIG. 6.

The product was completed by the following steps: 1) a composite material was prepared by using the decorative layer, shading layer, thermal insulating layer, non-woven fabric, PE film, medical adhesive layer and eyelash protective layer according to the above method, and the material was rolled into a coil, when being rolled, the shading layer was close to the heading; 2) the eyelash protective layer of the coil was cut off with a glasses shape semi-cutter (which can exactly cut off the eyelash protective layer without cutting the medical adhesive layer), the extra eyelash protective layer outside the glasses shape area was peeled off from the medical adhesive layer through a material receiving system, and the exposed medical adhesive layer and the isolation layer were compounded together; 3) for the compounded material obtained in step 2), a semi-cutter with the dolphin shape was used, the position (ensuring that cutting off the central position of the profile in step 3) after cutting off the eyelash protective layer adhered to the medical adhesive layer in step 2)) was adjusted to cut off the decorative layer, shading layer, thermal insulating layer, non-woven fabric, PE film and medical adhesive layer without cutting the isolation layer (as shown in FIG. 6), the composite material with dolphin shape was left on the surface of the isolation layer, and other materials were peeled off from the isolation layer through the material receiving system, and then the isolation layer was cut through the semi-cutter to obtain the product.

The thermal insulating layer of the light-proof eye protection paste in this example has a dual structure of IXPE foam and hydrogel, which has a better thermal insulation effect compared with the single-layer foam thermal insulating layer, and has a better protective effect on the strong light with high thermal effect such as laser, so as to avoid skin burns. Compared with the product of Example 6, the product is more firmly fixed.

1. A light-proof eye protection paste, comprising a decorative layer, a shading layer, a thermal insulating layer, a medical adhesive layer, an eyelash protective layer and an isolation layer from outside to inside, wherein the decorative layer is located at an outermost layer for aesthetic decoration, the shading layer is located inside the decorative layer and used for blocking light and preventing light from penetrating to hurt the skin, the thermal insulating layer is located inside the shading layer and used for absorbing the strong light energy transmitted to the shading layer, the medical adhesive layer is located inside the thermal insulating layer and used for sticking and fixing on the skin around eyes, the eyelash protective layer is located inside the medical adhesive layer and used for protecting eyelashes from sticking off by the medical adhesive layer, and the isolation layer is located inside the medical adhesive layer and the eyelash protective layer, and is used for protecting the unused medical adhesive layer.

2. The light-proof eye protection paste according to claim 1, wherein the decorative layer is a film with a thickness in a range of 0.01-0.5 mm, preferably in a range of 0.02-0.05 mm, and a color of the film can be any color, preferably transparent or translucent.

3. The light-proof eye protection paste according to claim 1, wherein the shading layer is a metal foil, and the metal foil can be any metal capable of forming a foil, preferably aluminum foil or copper foil.

4. The light-proof eye protection paste according to claim 1, wherein the shading layer is a metallized film, preferably an aluminized film.

5. The light-proof eye protection paste according to claim 1, wherein the shading layer has a thickness in a range of 0.01-0.2 mm, preferably in a range of 0.05-0.15 mm.

6. The light-proof eye protection paste according to claim 1, wherein the thermal insulating layer is a thermal poor conductor, with porous structure and soft texture, preferably foam or sponge structure.

7. The light-proof eye protection paste according to claim 1, wherein the thermal insulating layer is a thermal poor conductor, with film-like structure and soft texture.

8. The light-proof eye protection paste according to claim 1, wherein a material of the thermal insulating layer is selected from one of PU, EPDM, EPP, EVA and XPE/IXPE foam, with a thickness of 0.1-1.5 mm, preferably 0.5-0.8 mm.

9. The light-proof eye protection paste according to claim 1, wherein the thermal insulating layer is made of a non-transparent material, which configured to participate in blocking light at the same time of heat insulation, during IPL/LED/blue light treatments with low/no heat, the shading layer can be removed and the thermal insulating layer is used directly to block light.

10. The light-proof eye protection paste according to claim 1, wherein the thermal insulating layer has a dual structure composed of a smooth foam layer with poor thermal conductivity and a hydrogel layer with large specific heat capacity, the smooth foam layer is in contact with the shading layer, and the hydrogel layer replaces the medical adhesive layer to adhere to the skin.

11. The light-proof eye protection paste according to claim 1, wherein the thermal insulating layer has a dual structure composed of a smooth foam layer with poor thermal conductivity and a hydrogel layer with large specific heat capacity, the smooth foam layer is in contact with the shading layer, the other side of the hydrogel layer is a composite film of a non-woven fabric and an organic film with good compatibility, and the other side of the organic film of the composite film is coated with medical adhesive to adhere to the skin for fixation.

12. The light-proof eye protection paste according to claim 1, wherein the decorative layer film and the thermal insulating layer are fixed on both sides of the shading layer by laminating process.

13. The light-proof eye protection paste according to claim 1, wherein the decorative layer and the thermal insulating layer are compounded on both sides of the shading layer by the following steps: 1) keeping the film of the decorative layer and the materials of the shading layer even and non-folding under proper tension conditions; 2) coating a polyurethane adhesive on the surface of the decorative layer, after natural drying for 1-3 minutes, and compounding the decorative layer with the shading layer under the extrusion of a pressure roller; 3) repeating the above steps 1) and 2), and compounding the thermal insulating layer on the other side of the shading layer; 4) curing the polyurethane adhesive between the decorative layer, the shading layer and the thermal insulating layer for 5-36 hours under conditions of temperature 35-60° C. and humidity 35-60% to complete the compounding process.

14. The light-proof eye protection paste according to claim 1, wherein the medical adhesive layer comprises a safe, hypoallergenic and biocompatible medical adhesive, which can be one of medical hot-melt pressure-sensitive adhesive, acrylic pressure-sensitive adhesive and silicone gel, preferably acrylic pressure-sensitive adhesive.

15. The light-proof eye protection paste according to claim 1, wherein the eyelash protective layer is a soft, non-sticky, oval or round, biocompatible material with a thickness of 0.01-0.2 mm, preferably a film.

16. The light-proof eye protection paste according to claim 1, wherein the eyelash protective layer is located at the center of the medical adhesive layer, and has a smaller surface area than that of the medical adhesive layer, the edge area of the medical adhesive layer not covered by the eyelash protective layer has an enough width to fix the paste on the skin, the width is usually in a range of 3-15 mm, preferably 5 mm.

17. The light-proof eye protection paste according to claim 1, wherein the isolation layer is selected from one of silicone oil release paper, non-silicon release paper, fluorinated PET film, silicone oil PET film and PET film.

18. The light-proof eye protection paste according to claim 1, wherein the paste has a shape conforming to the structure and physiological characteristics of the eyes, and can be selected from one of symmetrical rain drop shape, bean sprout shape, oval shape, dolphin shape or conjoined glasses frame shape.

19. The light-proof eye protection paste according to claim 1, wherein the light-proof eye protection paste is symmetrically arranged and adhered on the rectangular isolation layer.

20. A method for preparing the paste light proof eye protection paste of claim 1, wherein the method comprises: 1) preparing a composite material by using the decorative layer, shading layer, thermal insulating layer, medical adhesive layer and eyelash protective layer, and rolling the material into a coil, when being rolled, the shading layer is close to the heading; 2) cutting off the eyelash protective layer of the coil with an oval or round semi-cutter (which can exactly cut off the eyelash protective layer without cutting the medical adhesive layer), peeling off the extra eyelash protective layer outside the central oval or round from the medical adhesive layer through a material receiving system, and compounding the exposed medical adhesive layer and the isolation layer together; 3) for the compounded material obtained in step 2), using the semi-cutter with the shape of claim 18, adjusting the position (ensuring that cutting off the central position of the profile in step 3) after cutting off the eyelash protective layer adhered to the medical adhesive layer in step 2)), cutting off the decorative layer, shading layer, thermal insulating layer and medical adhesive layer without cutting the isolation layer, leaving the composite material with specific shape on the surface of the isolation layer, and peeling off other materials from the isolation layer through the material receiving system, and then cutting the isolation layer by the semi-cutter to obtain the product.
